(19) United States
(12) Patent Application Publication
(10) Pub. No.: US 2008/0234689 A1
(43) Pub. Date: Sep. 25, 2008

(54) Vertebral Plate Measuring Device and Method of Use

(75) Inventors: Anthony J. Melkent, Memphis, TN (US); Douglas Dwayne Kave, Byhalia, MS (US)

Correspondence Address: COATS & BENNETT/MEDTRONIC, 1400 CRESCENT GREEN, SUITE 300, CARY, NC, 27518, US

(73) Assignee: Warsaw Orthopedic, Inc., Warsaw, IN (US)

(21) Appl. No.: 11/677,114

(22) Filed: Feb. 21, 2007

(51) Int. Cl. A61B 17/00; A61B 17/58

(52) U.S. Cl. 606/99; 606/205; 606/208

(57) ABSTRACT

The present application is directed to a surgical devices and methods to measure vertebral members for a vertebral plate. The device may include a handle with a first arm and a second arm. A trial plate may be attached to the handle. The trial plate may include a first trial plate section operatively connected to the first arm and a second trial plate section operatively connected to the second arm. Each of the first and second trial plate sections may include a distal side to contact against an outer side of the vertebral members. A scale may be operatively connected to the first and second trial plate sections and may include measurement indicia. The first and second arms may be movable to adjust a distance between the first and second sections and expose the measurement indicia to determine a size of the vertebral plate.

### BACKGROUND

The present application is directed to devices and methods for measuring a size of a vertebral plate required for a given anatomy and, more specifically, to adjustable devices and methods for measuring the size.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Discs are positioned in the intervertebral space between the vertebral members.

Various conditions may lead to damage of the vertebral members and/or discs. The damage may result from a variety of causes including a specific event such as trauma, a degenerative condition, a tumor, or infection. Damage to the discs and vertebral members can lead to pain, neurological deficit, and/or loss of motion.

Spinal plates have been developed for stabilization of various portions of the spine after various surgical procedures, particularly spinal fusion procedures. Conventional spinal fixation plates typically take the form of a unitary plate with a pair of bone screw openings at opposing ends. The plate is placed over an excavated bone graft-receiving site in the vertebral column, in which a bone graft is located for fusion to adjacent vertebrae, and secured in place with bone screws. The spinal plates fix the adjacent vertebrae on opposite sides of the bone graft-receiving site a set distance relative to one another.

In the past, the surgeon has been required to determine the required size of the spinal plate using a plurality of separate trials. The surgeon is required to individually handle the trials and insert them into the invertebral space until determining the correct size. This method is inconvenient for the surgeon as it requires preparing and handling the numerous trials.

### SUMMARY

The present application is directed to a surgical devices and methods to measure vertebral members for a vertebral plate. The device may include a handle with a first arm and a second arm. A trial plate may be attached to the handle. The trial plate may include a first trial plate section operatively connected to the first arm and a second trial plate section operatively connected to the second arm. Each of the first and second trial plate sections may include a distal side to contact against an outer side of the vertebral members. A scale may be operatively connected to the first and second trial plate sections and may include measurement indicia. The first and second arms may be movable to adjust a distance between the first and second sections and expose the measurement indicia to determine a size of the vertebral plate.

### BRIEF DESCRIPTION OF THE DRAWINGS

FIG. 1 is a schematic view of a device including a handle that is attachable to multiple trial plates according to one embodiment.

FIG. 2 is a side view of a device according to one embodiment.

FIG. 3 is a perspective view of trial plate sections mounted to the distal ends of a handle according to one embodiment.

FIG. 4 is a perspective view of mounts at the distal ends of the handle according to one embodiment.

FIG. 5 is perspective view of a trial plate including first and second sections according to one embodiment.

FIG. 6 is a perspective view of an attachment member on a trial plate section according to one embodiment.

FIG. 7 is a perspective view of a distal side of a trial plate including first and second sections according to one embodiment.

FIG. 8 is perspective view of a proximal side of a trial plate including first and second sections according to one embodiment.

FIG. 9 is a section view of a scale connected to a trial plate according to one embodiment.

FIG. 10 is a perspective view of a trial plate positioned between vertebral members according to one embodiment.

FIG. 11 is a schematic view of a handle according to one embodiment.

FIG. 12 is a perspective view of a distal side of a trial plate including first and second sections according to one embodiment.

FIG. 13 is a perspective view of a distal side of a trial plate including first and second sections according to one embodiment.

### DETAILED DESCRIPTION

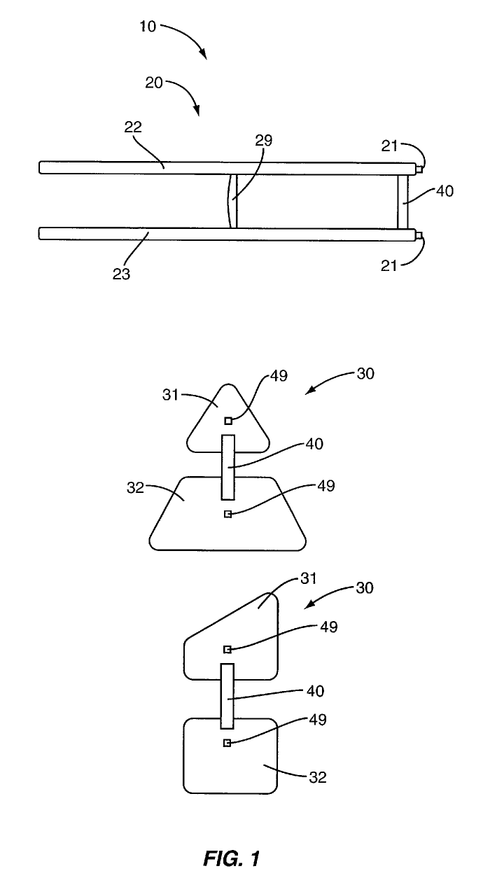

The present application is directed to devices and methods for measuring a plate size for a given anatomy of a patient. The devices are adjustable such that a single device may be used to measure the size without requiring multiple separate instruments. FIG. 1 schematically illustrates one embodiment of a device 10. The device generally includes a handle 20, one or more trial plates 30, and a scale 40. Handle 20 includes first and second arms 22, 23 that are attached together by a connector 29. Mounts 21 are positioned at the distal end of each arm 22, 23. The trial plates 30 include first and second sections 31, 32 each with an attachment member 49 to attach to the mounts 21. The first and second arms 22, 23 move about the connector 29 to adjust a distance between the trial plate sections 31, 32. A scale 40 measures the distance between the trial plate sections 31, 32. The scale 40 may be positioned at various locations, including between the arms 22, 23, or between the trial plate sections 31, 32. Various types and sizes of trial plates 30 may be attached to the handle 20 for measuring within the patient.

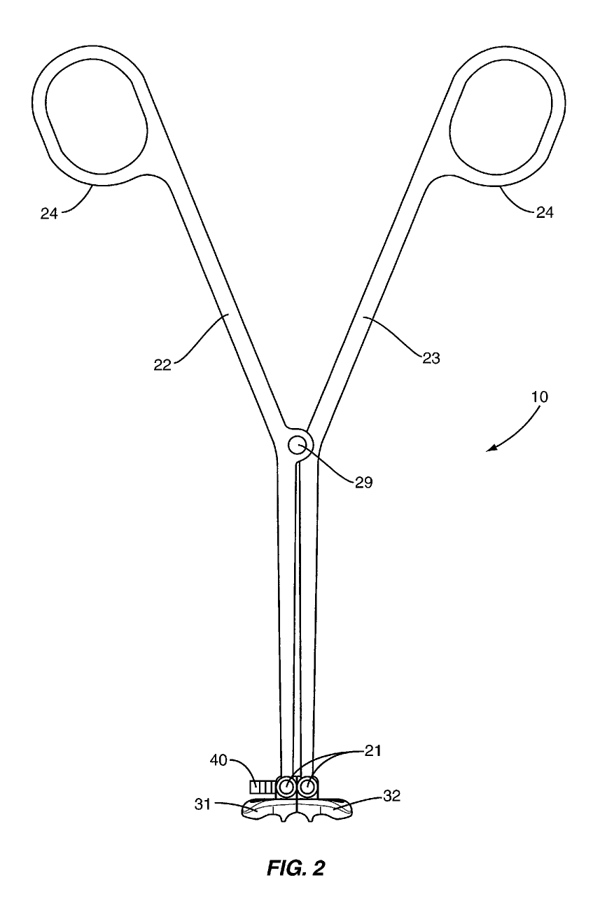

Handle 20 may include a variety of shapes and sizes. FIG. 2 illustrates one embodiment of the handle 20 with first and second arms 22, 23 that are pivotable about the connector 29. Proximal sections of each arm 22, 23 angle outward from the connector 29 and include a handle 24 for grasping and manipulating the device 10 by the surgeon. Distal sections of each arm 22, 23 are substantially straight from the connector 29 to the distal end. The handle 20 includes a length such that the proximal end remains outside of the patient while the distal ends are within the patient. In this embodiment, the arms do not cross at the connector 29. Accordingly, as the handles 24 of the arms 22, 23 move towards each other, the distal ends move apart from each other.

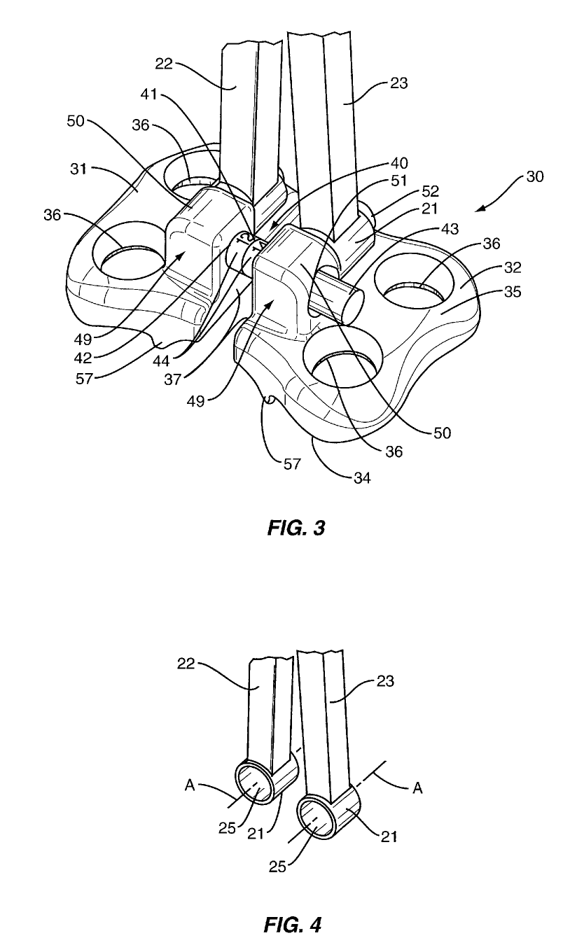

Mounts 21 are positioned at the distal end of each arm 22, 23. FIG. 3 illustrates the mounts 21 connected to trial plate sections 31, 32, and FIG. 4 illustrates the mounts 21 with the trial plate sections 31, 32 removed. In this embodiment, each mount 21 includes a cylindrical aperture 25 sized to receive the attachment member 49 on each trial plate section 31, 32. In one embodiment, an axis A of each aperture 25 is substantially parallel with an axis of the connector 29. This orientation provides for the trial plate 30 to be attached and detached from the handle 20 in a direction that is substantially perpendicular to the movement of the arms 22, 23.

Trial plates 30 are attached to the handle 20 to measure the anatomical size of the patient and the necessary size for a vertebral plate. As illustrated in FIG. 3, each trial plate 30 includes a first section 31 and a second section 32. The sections 31, 32 may be substantially the same shape and size, or may be different. The embodiment of FIG. 3 includes sections 31, 32 that are substantially the same. Each section 31, 32 includes a distal side 34 that faces towards the patient and a proximal side 35 that faces away from the patient. One or more apertures 36 extend through each section 31, 32. Each aperture 36 may be substantially the same, or may include different shapes and/or sizes.

The attachment members 49 are positioned on the proximal side 35 to connect with the handle 20. The attachment members 49 may be positioned at various locations about the proximal side 35 including aligned with the inner sides 37 of the sections 31, 32 as illustrated in FIG. 3, or spaced away from the inner sides 37. In one embodiment, one or both sections 31, 32 include two or more attachment members 49.

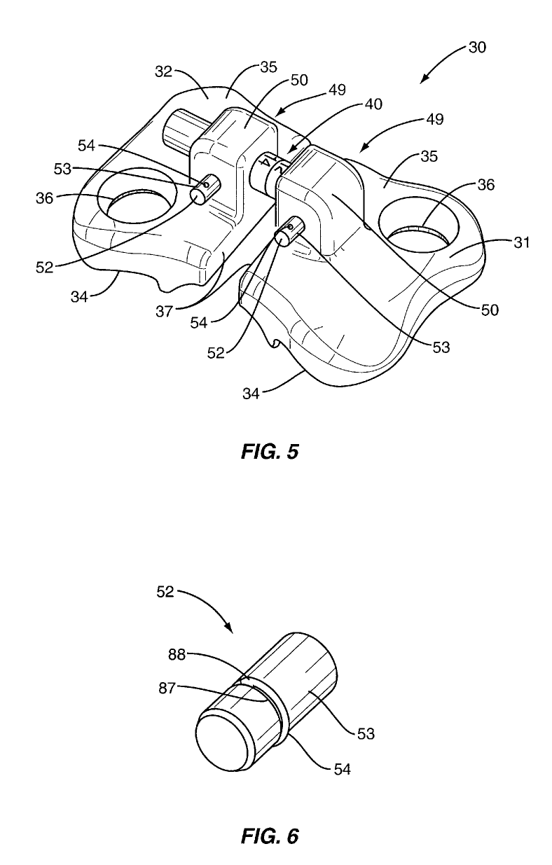

Attachment members 49 include a base 50 that extends outward from the proximal side 35. FIG. 5 illustrates one embodiment of the attachment members 49 that include the base 50 and extensions 52. The extensions 52 further include a neck 53 that extends outward from the base 50 and an outwardly-biased plunger element 54. The element 54 is biased outward beyond the surface of the neck 53 in a first orientation. During attachment of the trial plate 30, the element 54 is forced inward to a second orientation that allows passage of the mount onto the neck 53. After passing, the element 54 returns to the first orientation to capture the trial plate 30.

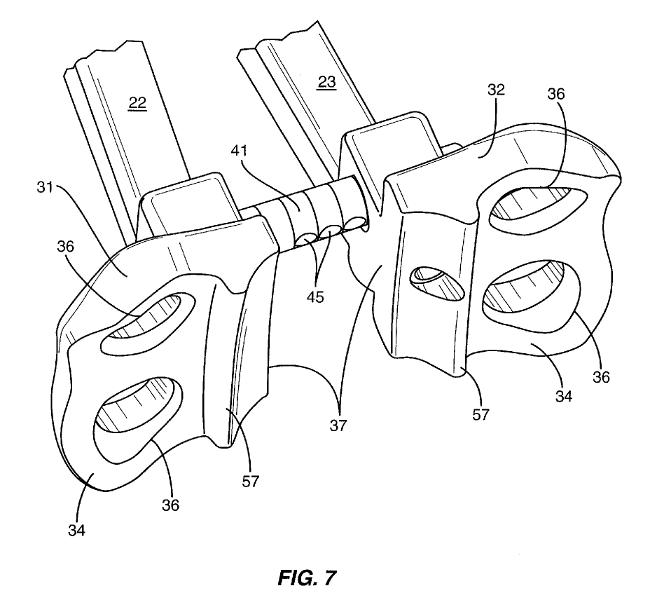

FIG. 6 illustrates another embodiment of the extension 52 with a neck 53 and a C-ring 88 positioned within a groove 87. This C-ring 88 extends outward beyond the surface of the neck 53 in the first orientation. During attachment of the trial plate 30, the C-ring 88 is compressed and moves inward to allow the trial plate 30 to move onto the neck 53. After passing, the C-ring 88 returns again and extends above the surface neck 53. The C-ring 88 may be constructed of a variety of flexible materials including plastics, and various metals such as steels.

In one embodiment, the distal side 34 of each trial plate section 31, 32 further includes a ridge 57 as illustrated best in FIG. 7. Ridge 57 extends outward from the distal side 34 and may extend along the entire width or a limited distance of each trial plate section 31, 32. In one embodiment, the ridges 57 are positioned in proximity to the inner sides 37 of the trial plate sections 31, 32. This positioning places the ridges 57 between the inner sides 37 and the apertures 36. In one embodiment, the ridges 57 may be substantially parallel to the inner sides 37. During the measuring process, the ridges 57 contact against the endplates 92 of the vertebral members 90 as will be explained in detail below.

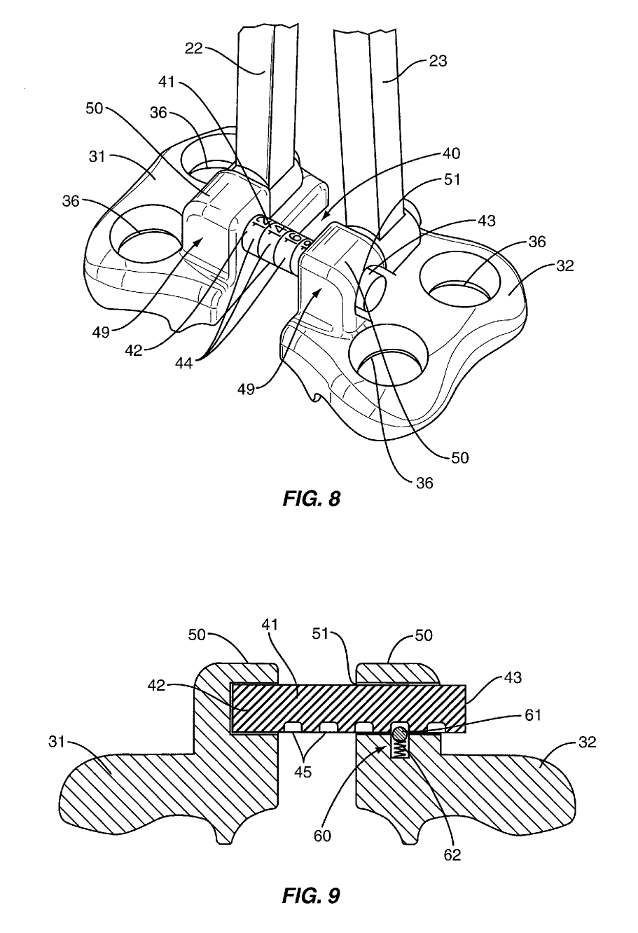

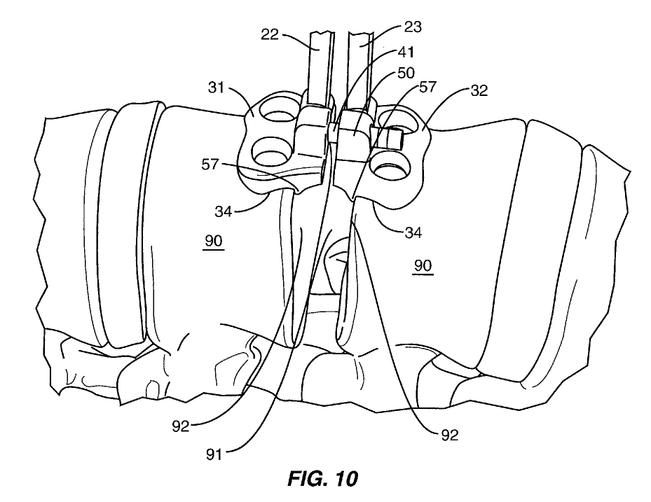

Scale 40 extends between the trial plate sections 31, 32. As illustrated in FIGS. 8 and 9, scale 40 includes an elongated member 41 with a first end 42 and a second end 43. The first end 42 is fixedly attached to the base 50 in the first trial plate section 31. The second end 43 is positioned within an aperture 51 in the base 50 of the second trial plate section 32. During movement of the handle arms 22, 23, the second end 43 moves within the aperture 51. Indicia 44 are positioned along length of the member 41 between the first and second ends 42, 43 and indicate the size of the trial plate 30. Indicia 44 may be positioned upwards towards the handle 20 to be visible to the surgeon during the measuring process as will be explained below.

Scale 40 may further include a mechanism to allow the trial plate sections 31, 32 to move in discrete lengths. As illustrated in FIGS. 7 and 9, cavities 45 may be positioned along a length of the member 41. In one embodiment, the cavities 45 are positioned on a bottom side of the member 41 to face in a direction towards the distal side 34 of the trial plate sections 31, 32. Cavities 45 are spaced apart with each cavity 45 aligned relative to a different marking 44. A plunger mechanism 60 is connected to the trial plate section 31. Plunger mechanism 60 includes a plunger element 61 that is connected with a biasing member 62. Plunger element 61 is sized to fit within each of the cavities 45 as the trial plate section 31 moves along the member 41.

The biasing member 62 forces the plunger element 61 upward into each of the passing cavities 45 in a ratchet-like operation. The force applied by the biasing member 62 is adequate to maintain the plunger element 61 in one of the cavities 45, but weak enough to be overcome by the force exerted by the handle 20. As the handle 20 moves to increase or decrease the space between the trial plate sections 31, 32, trial plate section 32 slides along the member 41. When the cavities 45 align with the plunger mechanism 60, the plunger element 61 is forced into the aligned cavity 45 by the biasing member 62. Additional movement of the handle 20 forces the plunger element 61 to recede out of the aligned cavity 45 thus allowing further movement of the trial plate section 32 along the elongated member 41.

The surgeon is able to use the scale 40 to determine the size of a vertebral plate. The distance is determined by the indicia 44 that are visible between the sections 31, 32. When the sections 31, 32 are in closer proximity as illustrated in FIG. 3, a limited number of indicia 44 are visible with the remainder being obscured by the base 50 on trial plate section 32. When the sections 31, 32 are spaced a greater distance apart, additional indicia are visible as illustrated in FIG. 8. The indicia 44 face upwards towards the surgeon to facilitate visualization. Further, indicia 44 may be color-coded with each increment indicated by a different color. The plunger mechanism 60 may also provide tactile feedback and give the surgeon some indication about the distance between the sections 31, 32.

FIG. 10 illustrates one embodiment of using the device 10 for measuring the vertebral anatomy of a patient. Prior to insertion into the patient, the surgeon determines the shape and size of the trial plate 30. The arrangement of the handle 20 and trial plates 30 provides for the use of a wide variety of trial plates 30. Once determined, the trial plate 30 is attached to the handle 20. The handle 20 is initially positioned in or towards a closed orientation with the trial plate sections 31, 32 in close proximity. The trial plate 30 is inserted into the patient with the distal sides 34 of the trial plate sections 31, 32 contacting the outer sides of the vertebral members 90. The trial plate sections 31, 32 overlap into the intervertebral space 91 with the ridges 57 positioned within the space 91.

The arms 22, 23 of the handle 20 are then manipulated to move the trial plate sections 31, 32 outward away from each other. The trial plate sections 31, 32 are moved outward until the ridges 57 contact against the endplates 92 of the vertebral members 90 as illustrated in FIG. 10. During the outward movement, base 50 of one of the trial plate sections 31, 32 moves along the length of the elongated member 41 thereby exposing the indicia 44 on the scale 40. The indicia 44 are positioned to face upward to be visible to the surgeon who can visually observe the size of the trial plate 30.

When the trial plate 30 is in the expanded orientation with the ridges 57 against the endplates 92, the surgeon is able to visually determine the sizing. In the embodiment with the plunger mechanism 60, the surgeon may also be able to tactilely feel the expansion of the trial plate 30 due to the plunger member 61 moving into and out of the cavities 45. The apertures 36 further align over the vertebral members 90 providing the surgeon with visual confirmation for placement of anchors.

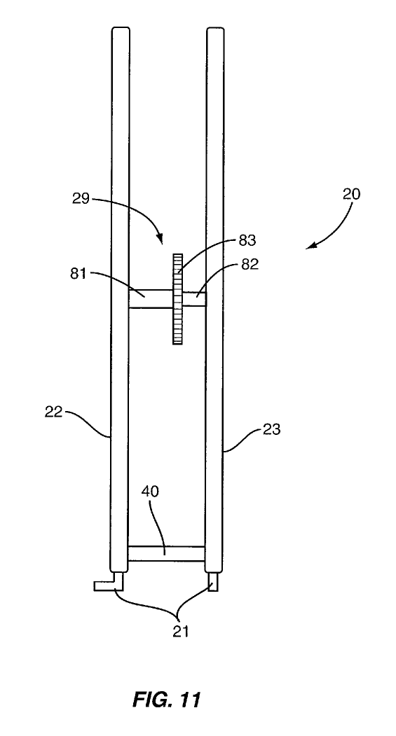

The device 10 may include various types of handles 20. In the embodiment described above, handle 20 included arms 22, 23 that pivot about an intermediate connector 29. In another embodiment, connector 29 is positioned towards a proximal end of the arms 22, 23. FIG. 11 illustrates another embodiment with a pair of arms 22, 23 connected by an intermediate connector 29. Connector 29 includes a first sleeve 81 that extends outward from the first arm 22 and is sized to receive a second sleeve 82 that extends outward from the second arm 23. A thumbwheel 83 is rotatably connected to the first sleeve 81 and includes a threaded aperture that aligns with the hollow interior of the sleeve 81. Rotation of the wheel 83 moves the second sleeve 82 into and out of the first sleeve 81 to adjust the distance between the arms 22, 23. In this embodiment, arms 22, 23 remain substantially parallel at the various orientations.

Various types of connections may be used for connecting the trial plate 30 to the handle 20. One embodiment described above includes the trial plate 30 including attachment members 49 that connect with apertures 25 in the handle 20. Other embodiments include but are not limited to a slot and groove arrangement, ramped features, ratchet teeth, over-center latching mechanism, Nitinol and shape-memory latches, and threaded pins. In the various embodiments, the features may be positioned on the handle 20 or on the trial plate 30. By way of example and using FIG. 3, the cylindrical apertures 25 may be positioned on the trial plate 20 and receive extensions positioned on the handle 20. Further, each arm 22, 23 may include a different type of connection mechanism. By way of example, first arm 22 may include a slot and groove arrangement, and the second arm 23 a threaded pin arrangement.

In one embodiment, the scale 40 extends between the trial plate sections 31, 32 as illustrated in FIGS. 3 and 8. In another embodiment, scale 40 is positioned between the arms 22, 23 of the handle 20. FIG. 1 schematically illustrates one embodiment with the scale 40 positioned towards the distal end of the arms 22, 23. The positioning of the arms 22, 23 coincide with the distance between the trial plate sections 31, 32. In one embodiment, the device 10 includes two or more different scales 40.

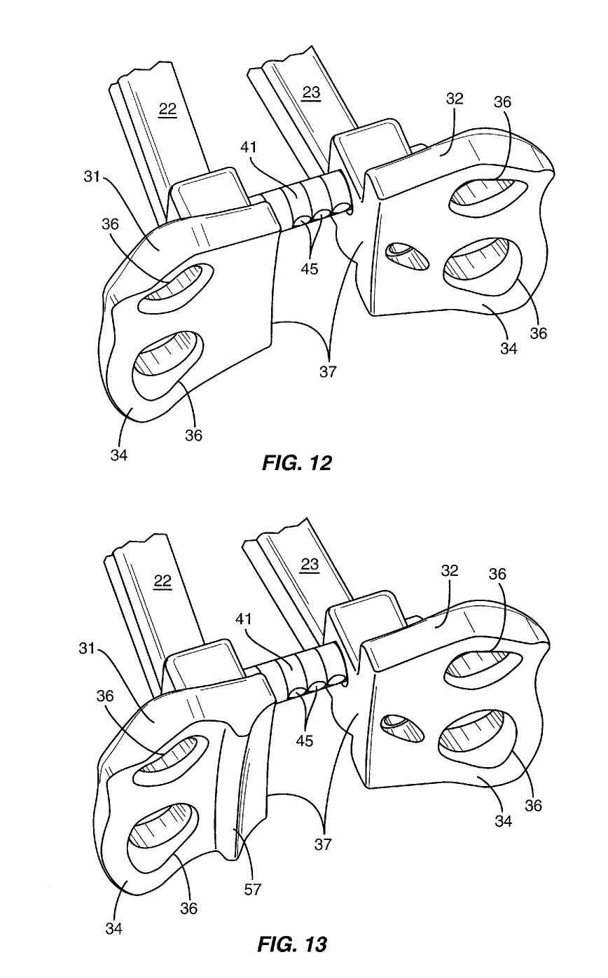

One embodiment of the trial plate 30 includes ridges 57 positioned on the distal side 34 of each trial plate section 31, 32. FIG. 12 illustrates another embodiment without ridges 57 on the distal sides 34. Alignment of the trial plate sections 31, 32 relative to the vertebral members 90 may performed visually by the surgeon.

FIG. 13 illustrates another embodiment with the first trial plate section 31 including a ridge 57 only on the first trial plate section 31. In this embodiment, second trial plate section 32 does not include a ridge. In one method of using this device, the first trial plate section 31 is aligned relative to the first vertebral member by contacting the ridge 57 against the endplate 92. The second trial plate section 32 is moved away and visually aligned with the second vertebral member.

One embodiment includes accessing the vertebral members 90 from an anterior approach to the spine. Other applications contemplate other approaches, including posterior, postero-lateral, antero-lateral and lateral approaches to the spine, and accessing other regions of the spine, including the cervical, thoracic, lumbar and/or sacral portions of the spine.

The term “distal” is generally defined as in the direction of the patient, or away from a user of a device. Conversely, “proximal” generally means away from the patient, or toward the user. Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A surgical device to measure vertebral members for a vertebral plate comprising:
a handle with a first arm and a second arm each including a proximal end and a distal end;
a first trial plate section attached to the distal end of the first arm and a second trial plate section attached to the distal end of the second arm, each of the trial plate sections including a distal side with a contact section that contacts against an outer side of the vertebral members;
a scale including an elongated member with visible measuring indicia, the scale being fixedly connected to the first trial plate section and movably connected to the second trial plate section;
the first and second trial plate sections being movable relative to each other between a first orientation with inner edges of each trial plate section being in proxmity and a second orientation with the inner edges being spaced apart and each aligned relative to one of the vertebral members;
the scale extending between the first and second sections to determine a size of the vertebral plate.

2. The device of claim 1, wherein the first and second trial plate sections each includes a ridge that extends outward from the contact section for positioning into an intervertebral space formed between endplates of the vertebral members in the first orientation and contacting against the endplates of the vertebral members in the second orientation.

3. The device of claim 1, wherein each of the first and second trial plate sections includes a pair of apertures that extend through the distal sides and proximal sides, the pairs of apertures being spaced away from the inner edges.

4. The device of claim 1, wherein the arms are pivotally connected together at a connector positioned between the proximal and distal ends.

5. The device of claim 1, wherein the arms remain substantially parallel during movement of the first and second trial plate sections between the first and second orientations.

6. The device of claim 1, wherein the elongated member includes a series of cavities and one of the first and second trial plate sections includes a plunger mechanism with a plunger element that fits individually within each of the series of cavities as the first and second trial plate sections move between the first and second orientations.

7. The device of claim 6, wherein the series of cavities are evenly spaced along a length of the elongated member.

8. The device of claim 1, wherein each of the first and second trial plate sections includes an attachment member with a base and an extension to be removably connected with mounts on the distal ends of the first and second arms.

9. The device of claim 1, wherein the first and second trial plate sections each have a substantially common shape.

10. The device of claim 1, wherein the first and second trial plate sections include different sizes.

11. The device of claim 1, wherein one of the first and second trial plate sections further includes a ridge that extends outward from the contact section for positioning into an intervertebral space formed between endplates of the vertebral members in the first orientation and contacts against one of the endplates of the vertebral members in the second orientation.

12. A surgical device to measure vertebral members for a vertebral plate comprising:
a handle with a first arm and a second arm;
a first trial plate section attached to the first arm and a second trial plate section attached to the second arm, each of the trial plate sections including a distal side that contacts against an outer side of the vertebral members and a proximal side that faces towards the handle;
a scale comprising an elongated member with measurement indicia extending along a length, the elongated member being fixedly connected to the first trial plate section and movably connected to the second trial plate section, the elongated member further including a series of cavities;
a plunger mechanism connected to the second trial plate section and including a plunger element sized to individually fit within each of the series of cavities;
the first and second arms be being movable to adjust a distance between the first and second trial plate sections with the measurement indicia being aligned relative to the second section, the movement of the first and second trial plate sections further causing the plunger element to move into and out each of the cavities.

13. The device of claim 12, wherein the elongated member is positioned on the proximal side of the first and second trial plate sections.

14. The device of claim 13, wherein a first end of the elongated member is positioned within a base that extends outward from the proximal side of the first trial plate section.

15. The device of claim 13, wherein a second end of the elongated member is movably positioned within a receptacle on the second trial plate member.

16. The device of claim 12, wherein each of the first and second trial plate members includes an inner edge that is aligned with endplates of the vertebral members.

17. The device of claim 12, further comprising ridges that extend outward from the distal side of each of the first and second trial plate members to contact against endplates on the vertebral members.

18. A surgical device to measure vertebral members for a vertebral plate comprising:
a handle with a first arm and a second arm each including a proximal end and a distal end;
a first trial plate section attached to the distal end of the first arm and a second trial plate section attached to the distal end of the second arm, each of the trial plate sections including a distal side with a contact section that contacts against an outer side of the vertebral members and a ridge that extends outward from the contact section for positioning into an intervertebral space formed between endplates of the vertebral members;
a scale with measuring indicia;
the first and second trial plate sections being movable between a first orientation with inner edges of each trial plate section being in contact and the ridges positioned within an interior of the intervertebral space, and a second orientation with the inner edges being spaced apart and the ridges in contact with the endplates of the vertebral members, the scale being positioned with the measuring indicia being visible.

19. The device of claim 18, wherein the scale is operatively connected to the first and second arms.

20. The device of claim 18, wherein the scale is operatively connected to the first and second trial plate sections.

21. The device of claim 18, further comprising a connector positioned along the handle to connect the first and second arms together.

22. A surgical device to measure vertebral members for a vertebral plate comprising:
a handle with a first arm and a second arm each including a proximal end and a distal end;
a trial plate including a first trial plate section operatively connected to the distal end of the first arm and a second trial plate section separate from the first section operatively connected to the distal end of the second arm, each of the first and second trial plate sections including a distal side with a substantially flat section that contacts against an outer side of the vertebral members, the first and second trial plate sections further including a proximal side that faces towards the handle;
a scale comprising an elongated member operatively positioned on the proximal side of the first and second trial plate sections, the elongated member including a first end fixedly connected to the first trial plate section and a second end positioned within a receptacle on the second trial plate section, the elongated member further including measurement indicia positioned between the first end and the second end;
the first and second arms be being movable to adjust a distance between the first and second sections and to move the elongated member within the receptacle with the indicia visible between the first and second sections.

23. The device of claim 22, wherein each of the first and second trial plate sections includes an outwardly-extending ridge that extends into an intervertebral space formed between endplates of the vertebral members.

24. The device of claim 22, wherein one of the first and second trial plate sections includes an outwardly-extending ridge that extends into an intervertebral space formed between endplates of the vertebral members.

25. A method of measuring vertebral members for a vertebral plate comprising:
positioning a trial plate mounted on a distal end of a handle into a patient and contacting a contact section on a distal side of the trial plate against an outer surface of first and second vertebral members;
positioning ridges that extend outward from the distal side within an intervertebral space formed between the first and second vertebral members;
manipulating the handle and separating a first trial plate section attached to a first arm of the handle from a second trial plate section attached to a second arm of the handle;
contacting a first ridge on the first trial plate section against an endplate of the first vertebral member and contacting a second ridge on the second trial plate section against an endplate of the second vertebral member; and
moving the second trial plate section along a scale that extends outward from the first trial plate section while separate the first and second trial plate sections and determining a size of the vertebral plate based on exposed measurement indicia that are visible on the scale between the first and second trial plate sections.

26. The method of claim 25, further comprising removing the trial plate from the distal end of the handle and attaching a second trial plate.

27. The method of claim 25, further comprising sliding the scale along a receptacle on the second trial plate section while separating the first and second trial plate sections.

28. The method of claim 25, further comprising moving a plunger element into and out of cavities positioned along the scale while separating the first and second trial plate sections.

29. A method of measuring vertebral members for a vertebral plate comprising:
positioning contact sections on distal sides of first and second trial plate sections against outer surfaces of first and second vertebral members;
separating the first and second trial plate sections; and
while separating the first and second trial plate sections, moving the second trial plate section along a scale that extends outward from the first trial plate section with a plunger element moving into and out of a series of cavities positioned within the scale.

30. The method of claim 29, further comprising positioning ridges that extend outward from the distal sides of each of the first and second trial plate sections against endplates on the first and second vertebral members.

31. The method of claim 29, further comprising moving the second trial plate section along the scale and exposing measurement indicia positioned on the scale.
